(19) United States
(12) Patent Application Publication
(10) Pub. No.: US 2013/0203980 A1
(43) Pub. Date: Aug. 8, 2013

(54) EXOPOLYSACCHARIDE OF SHIGELLA SONNEI BACTERIA, METHOD FOR PRODUCING SAME, VACCINE AND PHARMACEUTICAL COMPOSITION CONTAINING SAME

(71) Applicants: Petr Gennadievich Aparin, Odintzovo (RU); Vyacheslav Leonidovich Lvov, Moscow (RU); Stanislava Ivanovan Elkina, Moscow (RU); Marina Eduardovna Golovina, Moscow (RU); Vladimir Igorevich Shmigol, Moscow (RU)

(72) Inventors: Petr Gennadievich Aparin, Odintzovo (RU); Vyacheslav Leonidovich Lvov, Moscow (RU); Stanislava Ivanovan Elkina, Moscow (RU); Marina Eduardovna Golovina, Moscow (RU); Vladimir Igorevich Shmigol, Moscow (RU)

(21) Appl. No.: 13/877,305

(22) PCT Filed: May 6, 2011
(86) PCT No.: PCT/RU2011/000314

§ 371 Date: Apr. 1, 2013

(51) Int. Cl. C08B 37/00; C12P 19/26

(52) U.S. Cl. CPC C08B 37/006; C12P 19/26
USPC 536/53; 435/84

(57) ABSTRACT

For the first time, an O-specific polysaccharide antigen that is a Shigella Sonnei, phase I, exopolysaccharide has been produced and characterized, said exopolysaccharide being an authentic natural compound in the form of a bacterial capsular polysaccharide. The exopolysaccharide contains a non-toxic lipid component, namely non-hydroxylated fatty acids, and exhibits low pyrogenicity and high immunogenicity. Without using lipopolysaccharides as the source of production, an exopolysaccharide with a high degree of purity is produced from a liquid phase culture of S. sonnei bacteria by means of a workable industrial method with a high yield.

### CROSS-REFERENCE TO RELATED APPLICATIONS

This application claims priority to PCT patent application PCT/RU2011/000314 filed May 6, 2011.

### FIELD OF INVENTION

The invention relates to the clinical immunology and pharmacology, in particular it relates to the exopolysaccharide antigen of the bacteria Shigella sonnei, phase I-O-specific exopolysaccharide, the method of obtaining it, and the vaccine and pharmaceutical composition comprising it.

### BACKGROUND OF THE INVENTION

Almost 100 years now after discovering the bacillus Shiga, commonly known as Shigella dysenteriae, type 1, shigellosis is the one of the most important public health problems of almost all countries in the world. Annually, several hundred thousand children under the age of 5 die in developing countries from shigellosis caused by microorganisms of the genus Shigella. Outbreaks of shigellosis occasionally registered in developing countries of the northern hemisphere, caused by the bacteria S. sonnei, the only representative of group D, genus Shigella.

Relating to the aforementioned, WHO recommends as priority goal the development of a “global” anti-shigella vaccine, including protective compounds for pathogenic bacteria of genus Shigella, specifically S. sonnei, phase I (Kotloff K. L., Winickoff J. P, Ivanoff B., Clemens J. D., Swerdlow D. L., Sansonetti P. J., Adak G. K., Levine M. M. Global burden of Shigella infections: implications for vaccine development and implementation of control strategies. Bull. WHO, 1999, v. 77, p. 651-665).

Development of a monovaccine against shigellosis S.sonnei may be considered as a preliminary step for the solution of this general problem as an independent project extremely actual for many regions.

The specificity of immunity to Shigella infection is determined by the structure of the Shigella's main protective antigen—the polysaccharide O-antigen. Primary structure of O-specific polysaccharide obtained from the lipopolysaccharide (LPS) molecule of S. sonnei, phase I identified by Kenne et al (Kenne L., Lindberg B., Petersson K., Katzenellenbogen E., Romanowska E. Structural studies of the O-specific side-chains of the Shigella sonnei phase I lipopolysaccharide. Carbohydrate Res., 1980, 78:119-126).

O-antigen component of LPS is a polysaccharide composed of repeating disaccharide units of O-[4-amino-2-(N-acetyl)amino-2,4- dideoxy-β-D-galactopyranosyl]-(1→4)-[2-(N-acetyl)amino-2-deoxy-α-L-altrpyranuronic acid] linked by →(β) bonds to form a polysaccharide chain. This O-polysaccharide component of S. sonnei, phase I, covalently links to E.coli R2 type core domain, which, in turn, covalently links to lipid A and forming a linear molecule LPS.

Isolation of O-polysaccharide from the cell wall LPS does not represent significant technical difficulties. Thus, the method of isolation, first proposed by Freeman, includes the following main stages—obtaining culture of bacteria S. sonnei, phase I in liquid medium; separation of culture fluid from bacterial cells, extracting LPS from bacterial cell with aqueous phenol (Westphal O., Jann K. Bacterial lipopolysaccharide extraction with phenol: water and further application of the procedure. Methods Carbohydr. Chem., 1965, v. 5, p. 83-91); degradation of LPS with further isolation of the O-polysaccharide from it (Morrison D. C., Leive L. Fractions of lipopolysaccharide from Escherichia coli O111:B4 prepared by two extraction procedures. J. Biol. Chem. 250 (1975) 2911-2919).

Another method of obtaining highly purified O-specific antigen of Shigella sp is also known and includes the following stages: obtaining bacterial cultures in liquid medium; treatment of bacterial cultures with hexadecyltrimethylammonium bromide and subsequent extraction of LPS from bacterial cells; separation of LPS extract from bacterial cells; degradation of LPS with subsequent separation of O-polysaccharide (KR 20010054032 A). Thereby, all known methods of isolating O-specific antigens from Shigella sp. LPS are based on the stage of extraction, i.e. LPS extraction from bacterial cells, which causes the unavoidable loss of bacterial cell nativity.

It should be additionally marked, that the structure of O-specific antigens obtained by known methods from LPS's is determined by genomes of Shigella sp bacteria.

Practically all O-antigens obtained from Shigella sp. LPS's contain elements of core domain structures. Mild hydrolysis using 1% acetic acid is used for removal of lipid A from the LPS molecule, leads to obtaining a polysaccharide derivative, which is represented as a O-specific polysaccharide, connected to the “core” oligosaccharide (Fensom A. H., Meadow P. M. Evidence for two regions in the polysaccharide moiety of the lipopolysaccharide of Pseudomonas aeruginosa 8602. FEBS Lett. 9(2), 1970, 81-84; Morrison D C, Leive L. Fractions of lipopolysaccharide from Escherichia coli O111:B4 prepared by two extraction procedures. J Biol Chem. 250(8), (1975), 2911-19; Oertelt C, Lindner B, Skurnik M, Holst O. Isolation and structural characterization of an R-form lipopolysaccharide from Yersinia enterocolitica serotype O:8. Eur. J. Biochem. 2001 February; 268 (3), 554-64; Osborn M. J. Studies on the gram-negative cell wall. I. Evidence for the role of 2-keto-3-deoxyoctonate in the lipopolysaccharide of Salmonella typhimurium. Proc. Natl. Acad. Sci. USA, 50, (1963), 499-506).

It was proposed to use the O-polysaccharide from the LPS of the bacterial cell wall of S. sonnei, phase I, as a component of only conjugated vaccines against S.sonnei shigellosis, under it's covalent bonding with protein carriers—protein D Haemophilis influenzae, recombinant exoprotein A Pseudomonas aeruginosa (rEPA), recombinant diphtheria toxin (rDT), recombinant toxin B Clostriduum. difficile (rBRU) (US Pat. Appl. 2005/0031646; WO/2010/019890).

Investigations were conducted of the immunogenic and protective properties of conjugates containing O-polysaccharide from the LPS of the bacterial cells of Plesiomonas shigelloides O7, whose structure is identical to O-polysaccharide from LPS of bacteria S. sonnei, phase I, and proteins—exoprotein A P. aeruginosa (rEPA) or diphtheria toxoid CRM9 from mutant strain Corynobacterium diphtheriae (Cohen D., Ashkenazi S., Green M. S., Gdalevich M., Robin G., Slepon R., Yavzori M., Orr N., Block C., Ashkenazi I., Shemer J., Taylor D. N., Hale T. L., Sadoff J. C., Pavliakova D., Schneerson R., Robbins R. Double-blind vaccine controlled randomized efficacy trial of an investigational Shigella sonnei conjugate vaccine in young adults. Lancet, 1997, v. 349, pp. 155-159). It has been shown that the conjugate of O-polysaccharide with rEPA was immunogenic for experimental animals and humans when administered parenterally, causing in volunteers O-specific antibodies production and average level of protection against infection with efficacy coefficient of 74%. However, the rather short duration of the controllable experiment (2.5-7 months) is causing certain doubts in the rating for the protective potential of the vaccine. Recent immunogenicity trials on children of O-polysaccharide conjugate vaccine against S. sonnei infection based on rEPA-carrier revealed low immunogenicity of preparation for children of ages from 1 to 4 years (efficacy coefficient was 27.5%), as well as the early declining of immune response after immunization (Passwell J H, Ashkenzi S, Banet-Levi Y, Ramon-Saraf R, Farzam N, Lerner-Geva L, Even-Nir H, Yerushalmi B, Chu C, Shiloach J, Robbins J B, Schneerson R; Israeli Shigella Study Group. Age-related efficacy of Shigella O-specific polysaccharide conjugates in 1-4-year-old Israeli children. Vaccine. 2010, Mar., 2; 28(10), pp. 2231-2235).

Thus, the protein-polysaccharide conjugate vaccines against shigellosis S.sonnei have shown an insufficient immunogenicity in clinical trials on adults and children. It should be noted that the immunogenic properties of free, unconjugated O-polysaccharide from the LPS of the S. sonnei bacteria, phase I, as a vaccine immunogen is not known. Experimental data from Taylor et al show a practically full absence of immunogenic activity in mice against unconjugated polysaccharide from LPS of bacterial cells P. shigelloides, the structure of which is identical to that of S. sonnei, phase I O-antigen (Taylor D. N., Trofa A. C., Sadoff J., Chu C., Bryla D., Shiloach J., Cohen D., Ashkenazi S., Lerman Y., Egan W. Synthesis, characterization and clinical evaluation of conjugate vaccines composed of the O-specific polysaccharides of Shigella dysenteriae type 1, Shigella flexneri type 2a, and Shigella sonnei (Plesiomonas shigelloides) bound to bacterial toxoids. Infect. Immun., 1993, September, 61(9): 3678-3687).

Based on the aforementioned, the actuality of development of other approaches to the creation of O-antigen vaccines against S. sonnei infection is obvious. As alternative, perspective approach for development can be considered the creation of a unconjugated vaccine based on the O-antigen exopolysaccharide, produced by S. sonnei, phase I bacteria into the cultural medium. It is known, that many gram-positive and gram-negative bacteria produce not only polysaccharide components of cells, but also extracellular exopolysaccharides, which are secreted by the cell into the external medium and provide the protective function. Thus, the produced exopolysaccharides can be found both in a free state or form an extracellular capsule or microcapsule.

Sometimes exopolysaccharides produced by cells into the external medium represent specific highly-immunogenic antigens—potent inducers of protective antibody synthesis. Thus, a variety of such polysaccharide antigens are used in the vaccine compositions for prevention of infections, caused by meningococcus groups A and C, typhoid bacteria (Lindberg A. A. Polyosides (encapsulated bacteria). C. R. Acad. Sci. Paris, 1999, v. 322, p. 925-932).

Polysaccharide vaccine immunogenicity is determined by the primary structure of the polysaccharide antigen, its molecular mass, and ability to form aggregate structures (The vaccine book. Edited by B. R. Bloom, P.-H. Lambert Academic Press, San Diego 2003, pp. 436). At the same time, the primary structure of bacterial exopolysaccharide can be similar to or differ from that of O-specific polysaccharide domain from the cell wall LPS. (Goldman R. C., White D., Orskov F., Orskov I., Rick P. D., Lewis M. S., Bhattacharjee A. K., Leive L. A surface polysaccharide of Esherichia coli O111 contains O-antigen and inhibits agglutination of cells by anti-O antiserum. J. Bacteriol., 1982, v. 151, p. 1210-1221).

However, neither the primary structure of the exopolysaccharide of bacteria S. sonnei, phase I, nor its physico-chemical, immunobiological, and protective properties, nor the method of its isolation, nor even the fact of its existence are described in the literature.

The literature sources also do not describe the pharmaceutical compositions based on S. sonnei, phase I polysaccharides, the development of which can make significant contributions to clinical pharmacology. It only describes the usage of fragments of polysaccharides from LPS of S. sonnei, phase I cells, including from 1 to 5 disaccharide units, as nutrient supplement for oral administration, stimulating immune system development in infants between 1 and 6 months of age, determined by the increase of type1 T-helpers (Th1 response) to the type 2 T-helpers (Th2 response) ratio (US Pat. Appl. 2009/0317427 A1).

### SUMMARY

The objective of the claimed invention is to obtain, through a high-tech method, exopolysaccharides of bacteria S. sonnei, phase I, and develop on its basis a polysaccharide vaccines and pharmaceutical compositions.

The technical results, provided by the claimed inventions, are: (a) obtaining native polysaccharide from S. sonnei, phase I bacteria of high purity with a high yield on a commercial scale; (b) increasing the specificity, immunogenicity, protective activity and safety of developed vaccines; (c) high efficacy and broad spectrum of activity of the proposed pharmaceutical compositions.

For the first time is obtained a new polysaccharide antigen—exopolysaccharide, or capsular polysaccharide, secreted by S. sonnei, phase I bacteria into the external medium. In contrast to O-specific polysaccharide from LPS bacterial cell wall, an artificially isolated fragment of the molecule, the exopolysaccharide is an authentic natural compound, derived using S. sonnei bacteria, but without the use of LPS as its source. The primary structure of the exopolysaccharide was identical to that of the O-polysaccharide from LPS of bacteria S. sonnei, phase I, i.e. the exopolysaccharide consists of 1-100 repeating disaccharide units of O-[4-amino-2-(N-acetyl)amino-2,4- dideoxy-β-D-galactopyranosyl]-(1→4)-O-[2-(N-acetyl)amino-2-deoxy-α-L-altrpyranuronic acid] connected by (1→3) bonds to form a polysaccharide chain (FIG. 1 and FIG. 2). In contrast to the O-polysaccharide from bacterial cell LPS, the native exopolysaccharide includes a non-toxic lipid component, the composition of which contains non hydroxylated fatty acids with 16-18 carbon atoms in the molecule (FIG. 3, FIG. 4). The fatty acid content in it is no less than 0.01 (w/w) percent. Additionally, obtained by any method the exopolysaccharide from S. sonnei bacteria does not include elements of LPS core domain structure (FIG. 4). Exopolysaccharide can be prepared by any method, including genetic engineering, using the genome of S. sonnei bacteria. Preferably the exopolysaccharide is produced using S. sonnei bacteria by a method, including: (a) production of the bacterial culture in liquid phase; (b) separating the liquid phase from bacterial cells; (c) isolating the polysaccharide from liquid phase. Meanwhile, to avoid destroying the cell wall and LPS entry into the liquid phase, separating it from the bacterial cells is advisable to preserve the nativity of bacterial cells. Isolating the polysaccharide from the liquid phase can be carried out by a method comprising: (i) removing proteins and nucleic acids from the liquid phase; (ii) ultrafiltration and (iii) dialysis of obtained solution.

Obtained using the above method exopolysaccharide contains no more than 1% (w/w) of protein and 2% (w/w) of nucleic acid. The molecular weight of the polysaccharide, measured by gel filtration, is from 0.4 to 400 kDa. The main fraction of the exopolysaccharide is a biopolymer with molecular weight over 80 kDa (FIG. 5B), while the main fraction of O-polysaccharide has a molecular weight of not more than 26 kDa (FIG. 5A). Exopolysaccharide is immunogenic and causes mucosal protection from shigellosis S.sonnei by inducing synthesis of a specific antibodies against S. sonnei, phase I bacteria in mammalian organisms, including humans (Example 1C, FIG. 6; Examples 2D, 2F).

As noted above, the immunogenicity of the polysaccharide antigen is determined by its molecular weight, the ability to form aggregate structures, so the highest immunogenicity is found out for exopolysaccharide fraction with molecular weight from 80 to 400 kD. Immunogenicity of the high molecular weight fraction of the exopolysaccharide exceeds more than 7 times the immunogenicity of the O-polysaccharide from bacterial cells LPS (Example 1C, FIG. 6), it is apparently determined by the presence in the molecule of a non-toxic lipid component—a non hydroxylated fatty acid contributing to supramolecular aggregate structures formation. Additionally, the exopolysaccharide is apyrogenic for rabbits when administered intravenously at a dose of no more than 0.050 mcg/kg in a rabbit pyrogenicity test (Example 1D). Exopolysaccharide vaccine formulation meets WHO Expert Committee requirements for polysaccharide vaccines pyrogenicity parameter (WHO TR—WHO Technical report No. 840, 1994).

The claimed method for producing S. sonnei, phase I bacteria exopolysaccharide includes: (a) producing cultures of S. sonnei bacteria in liquid phase; (b) separating liquid phase from bacterial cells; (c) isolating polysaccharide from liquid phase. At the same time, the liquid phase, which maintains cell cultures viability, can be represented by a cultural medium of various composition and properties. Separating liquid phase from bacterial cells is preferably carried out while maintaining nativity of bacterial cells.

Thus, the claimed method for producing a polysaccharide, which excludes the use of LPS as its source, does not contain the stage of LPS extraction from bacterial cell walls, resulting in the inevitable loss of bacterial cell nativity.

Isolation of polysaccharide from liquid phase can be carried out by a method comprising: (i) removal of proteins and nucleic acids from liquid phase; (ii) ultrafiltration and (iii) dialysis of obtained solution.

The claimed vaccine for prophylaxis and/or treatment of S. sonnei shigellosis contains prophylactically and/or therapeutically effective amounts of S. sonnei, phase I bacteria polysaccharides, consisting of 1-100 repeating disaccharide units of O-[4-amino-2-(N-acetyl)amino-2,4- dideoxy-β-D-galactopyranosyl]-(1→4)-O-[2-(N-acetyl)amino-2-deoxy-α-L-altrpyranuronic acid] connected by (1→3) bonds to form a polysaccharide chain, and obtained using S. sonnei bacteria, but without the use of lipopolysaccharides as its source.

This polysaccharide is an exopolysaccharide, or capsular polysaccharide, secreted into the cultural medium by S. sonnei, phase I bacteria. The native exopolysaccharide includes a non-toxic lipid component, presented by non hydroxylated fatty acids from 16-18 carbon atoms in the molecule (FIG. 4). Its fatty acid content is less than 0.01% (w/w). Additionally, independently from the method of preparation with use S. sonnei bacteria, the polysaccharide does not include elements of the structure of LPS core domain (FIG. 4).

Exopolysaccharide can be prepared by any method, including genetic engineering, using the genome of S. sonnei bacteria. Preferably the exopolysaccharide is produced using S. sonnei bacteria by a method comprising: (a) producing bacterial culture in liquid phase; (b) separating the liquid phase from bacterial cells; (c) isolating the polysaccharide from liquid phase. Meanwhile, in order to avoid destroying the cell walls and LPS entry into the liquid phase, separation it from the bacterial cells is advisable to carry out under conditions for maintain the nativity of bacterial cells. Isolating the polysaccharide from the liquid phase can be carried out by a method comprising: (i) removing proteins and nucleic acids from the liquid phase; (ii) ultrafiltration and (iii) dialysis of obtained solution.

Obtained using the above method exopolysaccharide contains no more than 1% (w/w) of protein and 2% (w/w) of nucleic acid. The molecular weight of the polysaccharide, which is measured by gel filtration, is varied from 0.4 to 400 kDa. The main fraction of the polysaccharide is a biopolymer with molecular weight over 80 kDa (FIG. 5B).

Exopolysaccharide is immunogenic and causes mucosal protection from S. sonnei shigellosis by inducing synthesis of a specific antibodies against S. sonnei, phase I bacteria in mammalian organisms, including humans (Example 1C, FIG. 6; Examples 2D, 2F).

The highest immunogenicity is found out for exopolysaccharide fraction with molecular weight from 80 to 400 kDa. Immunogenicity of the high molecular weight fraction of the exopolysaccharide exceeds more than 7 times the immunogenicity of the O-polysaccharide from bacterial cell LPS (Example 1C, FIG. 6). The exopolysaccharide is apyrogenic for rabbits when administered intravenously at a dose of no more than 0.050 mcg/kg in a rabbit pyrogenicity test (Example 1D).

The claimed vaccine may comprise pharmaceutically acceptable additives, which may include pH stabilizers, preservatives, adjuvants, isotonizing agents or combinations of them. This vaccine may include exopolysaccharides in conjugated as well as unconjugated form. Meanwhile, the vaccine, comprised of the conjugated form of the polysaccharide, also contains carrier protein, namely diphtheria toxoid or tetanus toxoid, or P. aeruginosa protein A, or other proteins.

The claimed pharmaceutical composition contains effective amounts of S. sonnei, phase I bacteria polysaccharides, consisting of 1-100 repeating disaccharide units of O-[4-amino-2-(N-acetyl)amino-2,4-dideoxy-β-D-galactopyranosyl]-(1→4)-O-[2-(N-acetyl)amino-2-deoxy-α-L-altrpyranuronic acid] connected by (1→3) bonds to form a polysaccharide chain, and obtained using S. sonnei bacteria, but without the use of lipopolysaccharides as its source.

This polysaccharide is an exopolysaccharide, or capsular polysaccharide, secreted into the cultural medium by S. sonnei, phase I bacteria. The native exopolysaccharide includes a non-toxic lipid component, presented by non hydroxylated fatty acids from 16-18 carbon atoms in the molecule (FIG. 4). Its fatty acid content is less than 0.01% (w/w). Additionally, independently from the method of preparation with use S. sonnei bacteria, the polysaccharide does not include elements of the structure of LPS core domain (FIG. 4).

Exopolysaccharide can be prepared by any method, including genetic engineering, using the genome of S. sonnei bacteria. Preferably the exopolysaccharide is produced using S. sonnei bacteria by a method comprising: (a) producing bacterial culture in liquid phase; (b) separating the liquid phase from bacterial cells; (c) isolating the polysaccharide from liquid phase. Meanwhile, in order to avoid destroying the cell walls and LPS entry into the liquid phase, separation it from the bacterial cells is advisable to carry out under conditions for maintain the nativity of bacterial cells. Isolating the polysaccharide from the liquid phase can be carried out by a method comprising: (i) removing proteins and nucleic acids from the liquid phase; (ii) ultrafiltration and (iii) dialysis of obtained solution.

Obtained using the above method exopolysaccharide contains no more than 1% (w/w) of protein and 2% (w/w) of nucleic acid. The molecular weight of the polysaccharide, which is measured by gel filtration, is varied from 0.4 to 400 kDa. The main fraction of the polysaccharide is a biopolymer with molecular weight over 80 kDa (FIG. 5B).

Exopolysaccharide is the immune system response modulator in mammals, including humans (Example 3B). The exopolysaccharide is apyrogenic for rabbits when administered intravenously at a dose of no more than 0.050 mcg/kg in a rabbit pyrogenicity test (Example 1D).

The claimed pharmaceutical composition may comprise pharmaceutically acceptable targeted additives, which may include preservatives, stabilizers, solvents or combinations of them.

The claimed pharmaceutical composition can have a wide range of pharmacological activity and exhibits, in particular, an effective therapeutic antiviral effect under infection caused by influenza A virus subtype H1N1 (Example 3B, FIG. 9)

Also claimed is the use of polysaccharide from S. sonnei, phase I bacteria for production of vaccine or pharmaceutical composition. The stated polysaccharide consists of 1-100 repeating disaccharide units of O-[4-amino-2-(N-acetyl)amino-2,4-dideoxy-β-D-galactopyranosyl]-(1→4)-O-[-2-(N-acetyl)amino-2-deoxy-α-L-altrpyranuronic acid] connected by (1→3) bonds to form a polysaccharide chain, and obtained using S. sonnei bacteria, but without the use of lipopolysaccharides as its source.

This polysaccharide is an exopolysaccharide, or capsular polysaccharide, secreted into the cultural medium by S. sonnei, phase I bacteria. The native exopolysaccharide includes a non-toxic lipid component, presented by non hydroxylated fatty acids from 16-18 carbon atoms in the molecule (FIG. 4). Its fatty acid content is less than 0.01% (w/w). Additionally, independently from the method of preparation with use S. sonnei bacteria, the polysaccharide does not include elements of the structure of LPS core domain (FIG. 4).

Exopolysaccharide can be prepared by any method, including genetic engineering, using the genome of S. sonnei bacteria. Preferably the exopolysaccharide is produced using S. sonnei bacteria by a method comprising: (a) producing bacterial culture in liquid phase; (b) separating the liquid phase from bacterial cells; (c) isolating the polysaccharide from liquid phase. Meanwhile, in order to avoid destroying the cell walls and LPS entry into the liquid phase, separation it from the bacterial cells is advisable to carry out under conditions for maintain the nativity of bacterial cells. Isolating the polysaccharide from the liquid phase can be carried out by a method comprising: (i) removing proteins and nucleic acids from the liquid phase; (ii) ultrafiltration and (iii) dialysis of obtained solution.

Obtained using the above method exopolysaccharide contains no more than 1% (w/w) of protein and 2% (w/w) of nucleic acid. The molecular weight of the polysaccharide, which is measured by gel filtration, is varied from 0.4 to 400 kDa. The main fraction of the polysaccharide is a biopolymer with molecular weight over 80 kDa (FIG. 5B).

Exopolysaccharide is immunogenic and causes mucosal protection from S. sonnei shigellosis by inducing synthesis of a specific antibodies against S. sonnei, phase I bacteria in mammalian organisms, including humans (Example 1C, FIG. 6; Examples 2D, 2F). Additionally, the exopolysaccharide is also a modulator of immune system response in mammals, including humans (Example 3B). The exopolysaccharide is apyrogenic for rabbits when administered intravenously at a dose of no more than 0.050 mcg/kg in a rabbit pyrogenicity test (Example 1D).

The exopolysaccharide is apyrogenic for rabbits at a dose of no more than 0.050 mg/kg in a pyrogenicity test in rabbits when administered intravenously (Example 1D). The produced vaccine and pharmaceutical composition are intended for parenteral, oral, rectal, intra-vaginal, transdermal, sublingual and aerosol administration to mammals, including humans.

### BRIEF DESCRIPTION OF DRAWINGS

The invention is illustrated by the following figures.

FIG. 1 shows the structural formula of the monomer unit of S. sonnei, phase I bacteria exopolysaccharide.

FIG. 2 shows C13 NMR-spectrum of S. sonnei, phase I bacteria exopolysaccharide.

FIG. 3 shows results of GC mass-spectrometry of S. sonnei, phase I bacteria LPS.

FIG. 4 shows results of GC mass-spectrometry of S. sonnei, phase I bacteria exopolysaccharide; arrows indicate nonhydroxylated fatty acid signals.

FIG. 5 shows graphs of molecular weight distribution of O-specific polysaccharide, isolated from S.sonnei, phase I bacteria (a) and S.sonnei, phase I bacteria exopolysaccharide (b). In this case, the vertical axis represents the values for ultraviolet absorption at a wavelength of 225 nm; the horizontal axis represents time in minutes.

FIG. 6 shows graphs of antibody production (15 days) after primary (a) and secondary (b) immunization of mice with preparations made with S. sonnei, phase I bacteria exopolysaccharides (lot 33) and O-polysaccharide from S. sonnei, phase I bacteria LPS, with dose of 25 micrograms per mouse. On the vertical axis are the values for serum titer dilution.

FIG. 7 shows graphs of binding of antibodies from rabbit monoreceptor serum to S. sonnei, phase I O-antigen, with samples: S. sonnei exopolysaccharide (lot 33 and 35); O-polysaccharide from S. sonnei bacteria LPS; Salmonella enterica sv typhimurium LPS; S. flexneri 2a LPS in ELISA test. On the horizontal axis shows the values of serum titer dilution and the vertical axis—the optical density of reaction color substrate (ortho-phenylenediamine) at a wavelength reading of 495/650 nm.

FIG. 8 shows a graph of antibody production (15 days) after primary (a) and secondary (b) immunization of mice with vaccine consisting of unconjugated form of S. sonnei, phase I bacterial exopolysaccharide and with vaccine of conjugated with S. sonnei bacteria exopolysaccharide (lot 33) tetanus toxoid (TT), at a dosage of 25 micrograms of exopolysaccharides per mouse. The vertical axis shows values for serum titer dilution.

FIG. 9 shows graphs of survival rates of two groups of mice, infected with a dose of LD 100 of virulent influenza strain A subtype H1N1. The first group (experimental) received daily injections of the pharmaceutical composition, at a dose of 100 micrograms of exopolysaccharides per mouse, the second group (control)—injections of saline solution.

### DETAILED DESCRIPTION OF THE PREFERRED EMBODIMENTS

### Example 1

Preparation and Characteristics of S. sonnei, Phase I Bacteria Exopolysaccharide

A. Exopolysaccharide Preparation

Exopolysaccharide is prepared using S. sonnei, phase I cells. Bacteria culture prepared in liquid phase by deep cultivation of S.sonnei in nutrient medium. Separation of liquid phase and bacterial cells performed by flow centrifuge (Westphalia) with cooling, in compliance with regimens for smooth deposition of cells for maintain of cell nativity. Exopolysaccharide is isolated from the liquid phase and purified by removing from it proteins and nucleic acids, followed by ultrafiltration and dialysis of obtained solution. For this purpose the liquid phase is concentrated and dialyzed using an installation for ultrafiltration (Vladisart, membrane exclusion limit 50 kDa). The dialysate is lyophilized, redissolved in 0.05 M Tris-buffer, pH=7.2, containing 0.01% CaCl2 and MgCl2, RNAse and DNAse is added in concentration 100 mcg/mL and 10 mcg/mL, respectively, and after 16 hours of stirring at 37° C. the reaction mixture was treated with proteinase K (20 mcg/mL) for 2 hours at 55° C. The resulting clear solution is subjected to ultrafiltration and dialysis using an installation for ultrafiltration (Vladisart, membrane exclusion limit 50 kDa). If necessary, the final solution may be lyophilized and purified exopolysaccharide may be obtained with yield of 60-80%. The exopolysaccharide obtained by the aforementioned method contains not more than 1% (w/w) protein, determined by the Bradford method (Bradford M. M. A rapid and sensitive method for the quantitation of microgram quantities of protein utilizing the principle of protein-dye binding. Anal. Biochem. 1976, v. 72, pp. 248-254), and not more than 2% (w/w) nucleic acid, determined by the Spirin method (Spirin A. S. Spectrophotometric determination of the total amount of nucleic acids. Biochemistry, 1958, v. 23, No 4, p. 656).

B. The Structure, Composition, and Physico-Chemical Properties of Exopolysaccharide

The S. sonnei, phase I exopolysaccharide structure was studied using C¹³ NMR spectroscopy. NMR-spectrometry performed by Bruker spectrometer, model DRX-500, with XWINNMR software and impulse sequences from the manufacturer.

Survey of spectra were conducted in D₂₀ (99:95%) with acetone as a standard (31.5 ppm for C¹³). High resolution mass-spectrometry with electrospray ionization and ion detection using ion-cyclotron resonance performed on a Bruker Daltonics spectrometer, model Apex II, with 7 Tesla magnet.

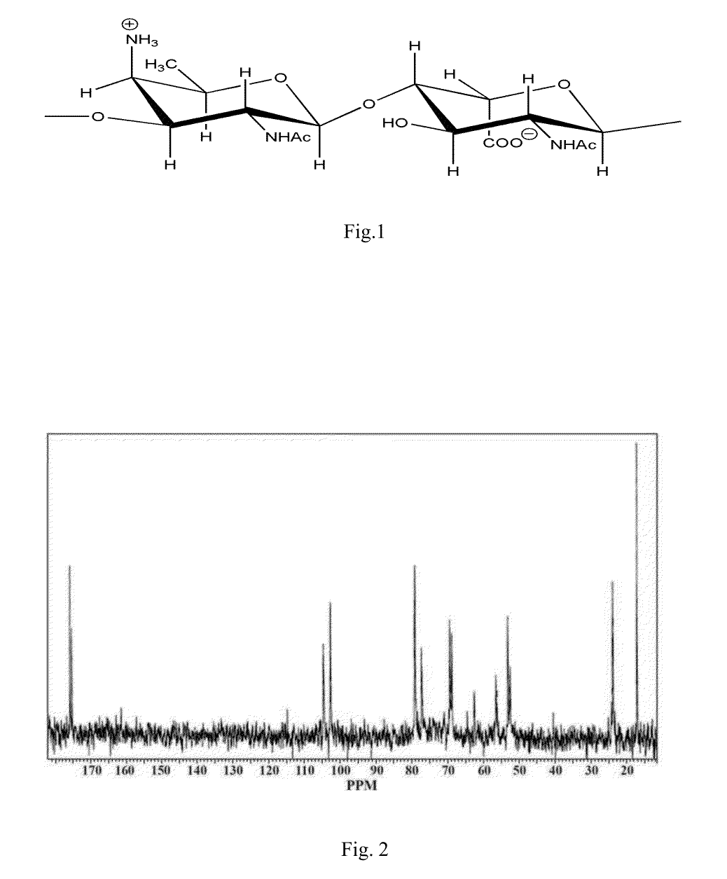

Comparative analysis of C¹³ NMR-spectrum of exopolysaccharide (FIG. 2) showed it's full identity to known C¹³ NMR-spectra of O-specific polysaccharide, isolated from LPS of S. sonnei, phase I, which clearly indicates identity of monomeric unit structure of both biopolymers (FIG. 1).

Studies of the exopolysaccharide's lipid component were carried out on the basis of fatty acid analysis using gas-liquid chromatography and GC/mass-spectrometry on Hewlett Packard, model 5890 chromatograph, connected to a NERMAG, model R10-10L mass spectrometer.

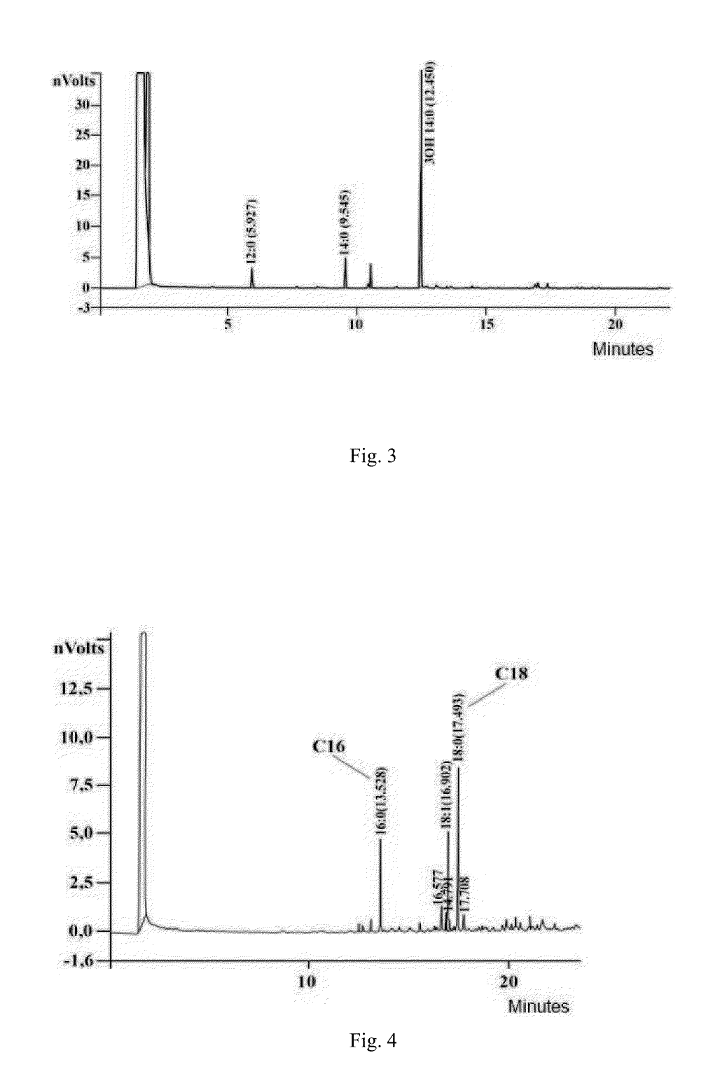

A comparative study of the fatty acid composition and exopolysaccharide structure and S. sonnei, phase I LPS is performed. Exopolysaccharide and LPS were subjected to methanolysis by treatment with 2M HCl/CH₃OH at 85° C. for 16 hours. Among methanolysis products of LPS are found lauric acid (12:0), myristic (14:0), and β-hydroxymyristic (30H14:0) acids (FIG. 3) whereas methanolysate of the exopolysaccharide contained, as basic products, methyl esters of higher fatty acids 16:0, 18:1, and 18:0.

The results of GC/mass spectrometry permit making the conclusion that the exopolysaccharide contains a non-toxic lipid component, composed of non hydroxylated fatty acids with 16-18 carbon atoms in the molecule, characteristic of diglycerides, in amounts no less than 0.01% (w/w). Exopolysaccharide, in contrast to LPS, did not contain oligosaccharide core components (heptose, Kdo) and lipid A (hydroxylated fatty acids) (FIG. 4).

Under mild acidic degradation of exopolysaccharide, cleavage of lipid part does not occur. Mild hydrolysis of LPS with 1% acetic acids leads to the removal of lipid A from LPS molecule. Meanwhile, the polysaccharide component obtained is an O-specific polysaccharide, linked to the core oligosaccharide (Fensom and Meadow, 1970; Morrison and Leive, 1975; Oertelt et al., 2001; Osborn, 1963).

Concluding, the exopolysaccharide is neither LPS, which must contain components core and lipid A domains, nor O-specific polysaccharide, which contain oligosaccharide fragment ‘core’, but is rather a glycoconjugate with another composition and structure, but with the same repeating monomer unit structure as S. sonnei O-antigen.

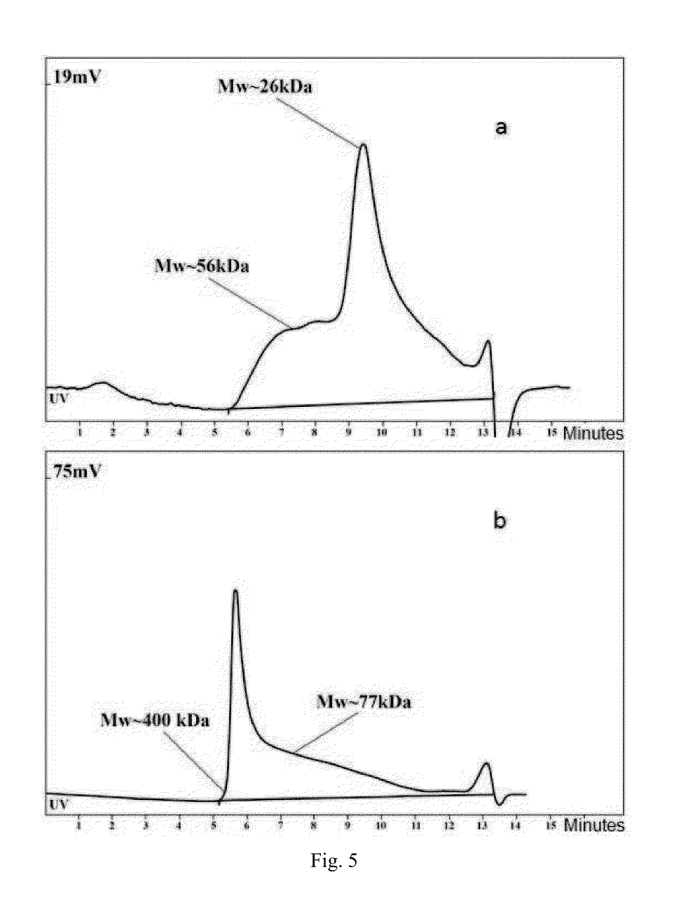

Study of molecular weight distribution of S.sonnei exopolysaccharide and O-specific polysaccharide, isolated from S.sonnei LPS, was performed by HPLC on a TSK 3000 SW with a flow-through UV detector (wavelength 225 nm) in a buffer, containing 0.02 M NaOAc, 0.2 M NaCl (pH 5.0). Comparative analysis of chromatograms of O-specific polysaccharide and exopolysaccharide show that the main fraction of the O-polysaccharide has a molecular weight of ˜26 kDa (FIG. 5), whereas the exopolysaccharide is a biopolymer with a molecular weight exceeding 80 kDa (FIG. 5B).

C. Exopolysaccharide Immunogenicity

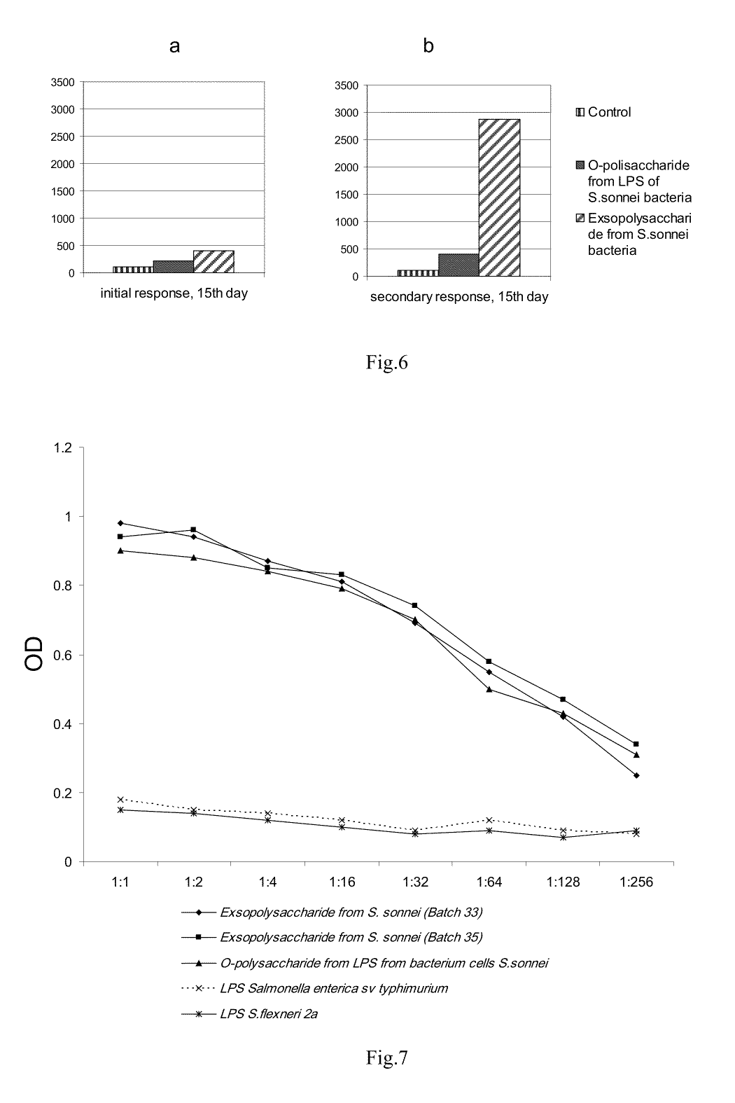

Two groups of mice strain (CBAXC57B1/6) F1 immunized intraperitoneally with S. sonnei, phase 1 bacteria exopolysaccharide drug preparation, lot 33, and O-polysaccharide preparation from S.sonnei, phase 1 bacterial cell LPS, with a dose of 25 micrograms per mouse. Exopolysaccharide drug preparation induced humoral immune response after a single dose injection and at day 15 the peripheral blood sera of animals is shown 3.4-fold increase in IgG antibodies; the O-polysaccharide preparation from bacterial cell LPS induced weak primary immune response—1.9-fold rise in of IgG antibodies levels on day 15, respectively (FIG. 6).

To study secondary immune response the same groups of mice were reimmunized with antigens at a dose of 25 micrograms per mouse a month after primary injection. On day 15, secondary response after repeated immunization with exopolysaccharide drug preparation, lot 33, 25-fold rise of IgG anti-O antibodies registered in mice, i.e. anamnestic secondary immune response was observed. After reimmunization with O-polysaccharide preparation from bacterial cell LPS, a low 3.4-fold increase in IgG anti-O antibodies was recorded in mice (FIG. 6). Thus, bacterial exopolysaccharide is much more immunogenic, inducing the formation of O-specific IgG antibodies, which has level 7 times higher than that induced by the O-polysaccharide of bacterial cell LPS.

D. Exopolysaccharide Pyrogenicity

The pyrogenicity of S. sonnei bacteria exopolysaccharide drug preparation (lot 33 and 35) drugs and O-polysaccharide from S. sonnei bacterial cell LPS was determined in comparison with pyrogenicity of LPS samples, extracted from cells of the same strain by Westphal method (Westphal O., Jann K. Bacterial lipopolysaccharide extraction with phenol: water and further application of the procedure. Methods Carbohydr. Chem., 1965, v. 5, pp. 83-91), and with commercial Vi-antigen vaccine. The test was conducted on Chinchilla rabbits weighing 2.8-3.05 kg in accordance with requirements of WHO Technical Regulations for Vi-polysaccharide vaccines (WHO Technical report No. 840, 1994). After administration of sample, rabbit rectal temperature was measured three times at 1 hour intervals. A drug was considered apyrogenic if total temperature increase did not exceed 1.15° C.

TABLE 1
Pyrogenicity of polysaccharide preparations and LPS from
S. sonnei bacteria and commercial Vi-antigen vaccine

| | Temperature | |
|---|---|---|
| Preparation | increase, in ° C. | Pyrogenicity |
| Vi-antigen typhoid vaccine | (0.1; 0.2; 0.3) | apyrogenic |
| [symbol] Vianvac [symbol] , | Σ: 0.6 | |
| lot 152 | | |
| Exopolysaccharide from | (0.2; 0.2; 0.1) | apyrogenic |
| S. sonnei bacteria, lot 33 | Σ: 0.5 | |
| Exopolysaccharide from | (0.2; 0.2; 0.3) | apyrogenic |
| S. sonnei bacteria, lot 35 | Σ: 0.7 | |
| O-polysaccharide from LPS of | (0.2; 0.1; 0.2) | apyrogenic |
| S. sonnei bacteria cells | Σ: 0.5 | |
| LPS from the cells of | (1.1; 0.8; 1.0) | high |
| S. sonnei bacteria | Σ: 2.9 | pyrogenicity |

Intravenous administration of S. sonnei bacterial exopolysaccharide drug preparation and O-polysaccharide from S. sonnei bacterial cell LPS at doses of 0.050 mcg per kg of body weight did not cause pyrogenic effect in rabbits. LPS, extracted from cells of the same strain, being a classic endotoxin, demonstrated high pyrogenicity.

### Example 2

Vaccines, Comprising of S. sonnei, Phase I Bacterial Exopolysaccharide

A. Use of the Exopolysaccharide for Production of Unconjugated Vaccine (Pharmaceuticals)

Preparation of unconjugated vaccine includes obtaining exopolysaccharide using S. sonnei, phase I bacteria in accordance with Example 1 (A) and subsequent aseptic filling of vials or syringes with solution containing the active substance and pharmaceutically suitable special additives, which may include pH stabilizers, preservatives, adjuvants, isotonizing agents or combinations thereof. Vaccination dose contains: unconjugated form of exopolysaccharide, in amount from 0.010 mg to 0.100 mg; phenol (preservative), not exceeding 0.75 mg, with addition of sodium chloride, dibasic sodium phosphate and monobasic sodium phosphate; sterile pyrogen-free water for injection, 0.5 mL.

B. Serological Activity of Unconjugated Vaccine

Serological activity and immune specificity of vaccine, including of exopolysaccharide in unconjugated form, in concentration of 100 mcg/mL (lots 33 and 35), were determined in inhibition passive hemagglutination reaction (IHA) in comparison with other O-antigens samples in concentration of 100 mcg/mL—O-polysaccharide from LPS of S. sonnei bacteria cells, as well as LPS's from S. sonnei, S. flexneri 2a, and Salmonella enterica sv typhimurium, obtained by Westphal method (Westphal O., Jann K. Bacterial lipopolysaccharide extraction with phenol: water and further application of the procedure. Methods Carbohydr. Chem., 1965, v. 5, p. 83-91). Commercial diagnostic kit contains S.sonnei antigen adsorbed erythrocytes (Microgen, Russia) and mono-receptor rabbit antiserum to S. sonnei O-antigen was used.

IHA concentration by vaccine, which includes exopolysaccharide (lots 33 and 35), O-polysaccharide from LPS, as well as S. sonnei bacterial LPS preparation, did not exceed 1.56 mcg/mL (Table 2). Heterologous bacterial LPS's of S.flexneri 2a and Salmonella enterica sv typhimurium had low serological activity in the IHA reaction with S.sonnei mono-receptor serum (inhibition concentration≧25 mcg/mL) (Table 2).

TABLE 2
IHA inhibition by unconjugated vaccine, includes exopolysaccharide
S. sonnei bacteria, and preparations of O-polysaccharide from LPS of
S. sonnei bacteria cells and LPS's from S. sonnei, S. flexneri 2a,
Salmonella enterica sv typhimurium bacteria

| | IHA concentration, |
|---|---|
| Preparation | mcg/mL |
| Vaccine, includes of S. sonnei | 1.56 |
| bacteria exopolysaccharide in | |
| unconjugated form (lot 33-1) | |
| Vaccine, includes of S. sonnei | 0.78 |
| bacteria exopolysaccharide in | |
| unconjugated form (lot 35-1) | |
| O-poly saccharidefrom LPS of | 1.56 |
| S. sonnei bacteria cells | |
| LPS of S. sonnei bacteria | 0.78 |
| LPS of S. flexneri 2a bacteria | 25.00 |
| LPS of Salmonella enterica | >25.0 |
| sv typhimurium bacteria | |

Interaction of in vitro the vaccine lots, includes unconjugated exopolysaccharide of S. sonnei bacteria at concentrations of 100 mcg/mL (lots 33-1 and 35-1), and other O-antigens in concentrations of 100 mcg/mL—O-polysaccharide from LPS of S. sonnei bacteria cells, LPS's from S.flexneri 2a and Salmonella enterica sv typhimurium bacteria, with rabbit mono-receptor serum antibodies to S. sonnei O-antigen is detected in ELISA test. Under solid phase absorption, the vaccine, includes of S. sonnei bacterial exopolysaccharide and O-polysaccharide sample from S. sonnei bacterial cell LPS, effectively interacted with S. sonnei O-antigen antiserum (FIG. 7).

C. Pyrogenicity of Unconjugated Vaccine

Pyrogenicity of vaccine, containing 100 mcg/mL of S. sonnei bacteria exopolysaccharide in the unconjugated form (lots 33 and 35), was determined in comparison with pyrogenicity of commercial Vi-antigen vaccine, O-polysaccharide from LPS of S. sonnei bacteria and LPS's isolated from cell culture supernatant and cells of the same strain using the Westphal method described in Example 1C. Test results are shown in Table 3.

TABLE 3
Pyrogenicity of the vaccine, containing S. sonnei bacteria
exopolysaccharide in the unconjugated form, commercial Vi-
antigen vaccine, preparations of O-polysaccharide from LPS of
S. sonnei bacteria and LPS's of S. sonnei bacteria

| | Temperature | |
|---|---|---|
| Preparation | increase, ° C. | Pyrogenicity |
| Vi-antigen typhoid vaccine | (0.3; 0.2; 0.0) | apyrogenic |
| [symbol] Vianvac [symbol] , lot 152 | Σ: 0.5 | |
| Vaccine, includes | (0.2; 0.2; 0.2) | apyrogenic |
| exopolysaccharide from | Σ: 0.6 | |
| S. sonnei bacteria, (lot 33-1) | | |
| Vaccine, containing | (0.2; 0.1; 0.3) | apyrogenic |
| exopolysaccharide from | Σ: 0.6 | |
| S. sonnei bacteria, (lot 35-1) | | |
| O-polysaccharide from LPS of | (0.1; 0.1; 0.3) | apyrogenic |
| S. sonnei bacteria cells | Σ: 0.5 | |
| LPS from supernatant of | (1.2; 1.2; 1.1) | highly |
| S. sonnei bacteria culture | Σ: 3.5 | pyrogenic |
| LPS from S. sonnei bacteria | (1.1; 0.9; 1.1) | highly |
| cells | Σ: 3.1 | pyrogenic |

Intravenous administration of vaccine, includes of S. sonnei bacteria exopolysaccharide, at a dose of 0.050 mcg per kg body weight did not cause pyrogenic effect in rabbits. Preparation containing LPS from S.sonnei bacteria cells of the same strain shown high pyrogenicity and thus represents a classic endotoxin.

D. Protective Properties of Unconjugated Vaccine

To study formation of protective mucosal immunity in guinea pigs, laboratory animals weighing 200-250 g were immunized with subcutaneous injection of vaccine, includes 100 mcg/mL of unconjugated S. sonnei bacterial exopolysaccharide (lots 33 and 35) and a preparation of O-polysaccharide from LPS of S. sonnei bacteria cells, in doses of 25 and 50 mcg per animal twice in the back region with 10 day interval. Control animals were given saline instead of the preparation. Ten days after the last immunization, S.sonnei kerato-conjunctivitis (Sereny test) was induced in the experimental and control animals by introduction into the eye conjunctiva cell suspension of virulent strain of S. sonnei in a dose, close to ID₁₀₀ (10⁹ cells), and in a dose close to 2ID₁₀₀(2×10⁹ cells), in 30 mcL of sterile saline. All control group animals, infected with a dose of 2×10⁹ cells, and 90% of control group animals, infected with a dose of 10⁹ cells, developed S. sonnei kerato-conjunctivitis (Table 4). Immunization with vaccine, includes of exopolysaccharide (lots 33 and 35), in a dose of 25 mcg provided eye protection rate 70-90% of experimental animals infected with a dose of 10⁹ cells; when infected with 2×10⁹ cells dose, eye protection rate varied from 50 to 70%, respectively. Higher dose of 50 mcg immunization with the same vaccine provided eye protection rate of 55 to 85% in experimental animals infected with a dose of 10⁹ cells; when infected with 2×10⁹ cells dose, eye protection level varied from 50 to 70%, respectively. Thus, under subcutaneous immunization of the animals with vaccine based on unconjugated form of S. sonnei bacterial exopolysaccharide (lots 33 and 35), a marked local anti-Shigella immunity was registered, meanwhile immunization with preparation of O-polysaccharide from LPS of S. sonnei bacterial cells did not shown anti-Shigella effect of the preparation.

TABLE 4
Protective mucosal immunity to infection S. sonnei in guinea pigs as a result of the systemic
immunization with vaccine, based on unconjugated form of S. sonnei bacteria exopolysaccharide

| | Preparation | Infection dose | | No. of | | No. of eyes | Rate of |
|---|---|---|---|---|---|---|---|
| | dose, | (No. of cells in | No. of | infected | No. of eyes | protected | the eye |
| | mcg per | 30 mcL of saline | infected | animal | with kerato- | from kerato- | protection, |
| Preparation | animal | solution) | animals | eyes | conjunctivitis | conjunctivitis | % |
| Vaccine, containing | 25 | 109 | 10 | 20 | 2 | 18 | 90 |
| exopolysaccharide | 25 | 2 × 109 | 10 | 20 | 6 | 14 | 70 |
| from S. sonnei | 50 | 109 | 10 | 20 | 9 | 11 | 55 |
| bacteria, (lot 33) | 50 | 2 × 109 | 10 | 20 | 10 | 10 | 50 |
| Vaccine, containing | 25 | 109 | 10 | 20 | 6 | 14 | 70 |
| exopolysaccharide | 25 | 2 × 109 | 10 | 20 | 10 | 10 | 50 |
| from S. sonnei | 50 | 109 | 10 | 20 | 3 | 17 | 85 |
| bacteria (lot 35) | 50 | 2 × 109 | 10 | 20 | 6 | 14 | 70 |
| O-polysaccharide | 25 | 109 | 10 | 20 | 12 | 4 | 20 |
| from LPS of S. sonnei | 25 | 2 × 109 | 10 | 20 | 14 | 6 | 0 |
| bacteria cells | 50 | 109 | 10 | 20 | 16 | 4 | 10 |
| | 50 | 2 × 109 | 10 | 20 | 17 | 3 | 15 |
| Control | — | 109 | 10 | 20 | 18 | 2 | 10 |
| | — | 2 × 109 | 10 | 20 | 20 | 0 | 0 |

E. Safety of Unconjugated Vaccine

Vaccine, including the unconjugated form of S. sonnei bacterial exopolysaccharide (lot 33), in a dose of 50 mcg of antigen, contained in 0.5 mL of phenol-phosphate buffer solution, and the product for comparison—typhoid Vi-antigen vaccine “Vianvac”, in a dose 25 mcg, were single injected subcutaneously to two groups of 20 adult volunteers in the upper third of the shoulder. Temperature reactions to the drug injection, general side effects and local reactions of volunteers were studied for the first three days after immunization. Vaccine, includes of S. sonnei bacterial exopolysaccharide (lot 33), administered in 50 mcg dose, showed high safety profile for adult volunteers. Temperature reactions in the 37.1-37.5° C. range were found in only 5% of volunteers, higher temperature reactions and general side effects were absent; local reaction (pain at injection site) was detected in only one volunteer (Table 5).

TABLE 5
Safety of the vaccine, includes the unconjugated
form of S. sonnei bacterial exopolysaccharide
under immunization of the adult volunteers

| | Vaccine, containing | |
|---|---|---|
| | exopolysaccharide | Vi-antigen vaccine |
| | from S. sonnei | [symbol] Vianvac [symbol] |
| Reactions on vaccine | bacteria (lot 33), | (lot 193), 25 mcg |
| administration | 50 mcg dose | dose |
| Temperature reactions | 5% of volunteers | 5% of |
| (37.1-37.5° C.) | | volunteers |
| Temperature reactions | absent | Absent |
| (37.6-38.5° C.) | | |
| Temperature reactions | absent | Absent |
| (38.5° C. and up) | | |
| Side effects | absent | Absent |
| Local reactions (pain) | 1 case | 1 case |

F. Immunogenicity of Unconjugated Vaccine

Immunogenicity of vaccine, including unconjugated S. sonnei bacterial exopolysaccharide (lot 33), for adult volunteers was determined in serological studies using tests: enzyme-linked immunosorbent analysis (ELISA) and passive hemagglutination reaction (PHA). Vaccines, includes of S. sonnei bacterial exopolysaccharide (lot 33), in a dose of 50 mcg of antigen, contained in 0.5 mL of phenol-phosphate buffer solution, and the product for comparison—typhoid Vi-antigen vaccine “Vianvac”, in 25 mcg dose, were single injected subcutaneously to two groups of 20 adult volunteers in the upper third of the shoulder. Blood sera for testing were taking from subject before vaccination and after 30 and 60 days after vaccination, respectively. To perform ELISA analysis, microplates coated with S. sonnei bacterial exopolysaccharide, rabbit antibodies against human IgG, IgM, IgA, conjugated with horseradish peroxidase (Sigma, USA) were used. The optical density was measured on a Bio-Rad iMark ELISA-reader under dual wavelength readings (490/630nm). PHA test was performed according to manufacturer's instructions, using S. sonnei commercial erythrocyte diagnosticum (Microgen, Russia).

Immunogenicity was evaluated according to following criteria: 4-fold seroconversion compared to background serum, level of antigenic response before and after vaccination; also geometric mean antibody titer (GM) was measured, titers fold rise in vaccinated group in comparing with background sera levels.

The increase in anti-O antibody titers was observed in all volunteers who were given vaccine with S. sonnei bacterial exopolysaccharide (lot 33). The high rises agglutinating antibody titer before and after vaccination was registered; 40.7× and 42.5× fold rise on 30th and 60th days after vaccination, respectively. High levels of seroconversion of antibodies to S. sonnei O-antigen, comprising ≧90% was registered among vaccinated subjects. In subjects immunized with “Vianvac” vaccine, rises in specific antibodies to exopolysaccharide and 4-fold seroconversions were not observed (Table 6).

High rises of antibody titers, especially IgA class, were revealed under the fold rise and seroconversion study of IgA, IgG, IgM classes of antibodies to S. sonnei O-antigen in ELISA test, compared to background level, among subjects immunized with vaccine, includes of S. sonnei bacterial exopolysaccharide (lot 33). Thus, fold rise of titer IgA antibodies on the 30th and 60th day after immunization was 25.7× and 30.2×; IgG antibodies—6.1× and 5.8×, respectively. Seroconversion rate of O-specific antibody IgA, IgG classes was high and consist of 95% and 95% for IgA; 75% and 70% for IgG on the the 30th and 60th days after vaccination, respectively. Therefore, the claimed vaccine, includes of unconjugated S. sonnei bacteria exopolysaccharide, under a single subcutaneous immunization adult volunteers, induces human systemic adaptive immune response with dominating antibody of IgA class.

TABLE 6
Induction systemic immune response in adult volunteers under a subcutaneous immunization
by vaccine, includes unconjugated S. sonnei bacteria exopolysaccharide

| | | | | | |
|---|---|---|---|---|---|
| | | | % of | | |
| | | Antibody | volunteers | Antibody | % of volunteers |
| | | titer fold | with 4-x fold | titer fold | with 4-x fold |
| | | rise 30 | seroconversion | rise 60 | seroconversion |
| Vaccine and the | No. of | days after | 30 days after | days after | 60 days after |
| immunization dose | volunteers | vaccination | vaccination | vaccination | vaccination |

PHA test-agglutinating antibodies

| | | | | | |
|---|---|---|---|---|---|
| Vaccine, includes | 20 | 40.7 | 90% | 42.5 | 95% |
| exopolysaccharide | | | | | |
| from S. sonnei | | | | | |
| bacteria, | | | | | |
| (lot 33), 50 mcg | | | | | |
| Vi-antigen vaccine | 20 | 1.14 | None | 1.16 | None |
| [symbol] Vianvac [symbol] (lot 193), | | | | | |
| 25 mcg | | | | | |

ELISA test - IgA

| | | | | | |
|---|---|---|---|---|---|
| Vaccine, includes | 20 | 25.7 | 95% | 30.2 | 95% |
| exopolysaccharide | | | | | |
| from S. sonnei | | | | | |
| bacteria, | | | | | |
| (lot 33), 50 mcg | | | | | |
| Vi-antigen vaccine | 20 | 0.82 | None | 0.99 | None |
| [symbol] Vianvac [symbol] (lot193), | | | | | |
| 25 mcg | | | | | |

ELISA test - IgG

| | | | | | |
|---|---|---|---|---|---|
| Vaccine, includes | 20 | 6.1 | 75% | 5.8 | 70% |
| exopolysaccharide | | | | | |
| from S. sonnei | | | | | |
| bacteria, | | | | | |
| (lot 33), 50 mcg | | | | | |
| Vi-antigen vaccine | 20 | 1.06 | None | 1.09 | None |
| [symbol] Vianvac [symbol] (lot193), | | | | | |
| 25 mcg | | | | | |

ELISA test - IgM

| | | | | | |
|---|---|---|---|---|---|
| Vaccine, includes | 20 | 2.51 | 50% | 2.73 | 50% |
| exopolysaccharide | | | | | |
| from S. sonnei | | | | | |
| bacteria, | | | | | |
| (lot 33), 50 mcg | | | | | |
| Vi-antigen vaccine | 20 | 1.10 | None | 1.14 | None |
| [symbol] Vianvac [symbol] (lot193), | | | | | |
| 25 mcg | | | | | |

G. Use of the Exopolysaccharide for Production of Conjugated Vaccine (Pharmaceuticals)

The exopolysaccharide is obtained using S. sonnei bacteria, phase in accordance with Example 1 (A). Obtaining conjugate of exopolysaccharide with protein can be performed by any of the known methods. In framework of this study, was used a method (Taylor D. N., Trofa A. C., Sadoff J., Chu C., Brula D., Shiloach J., Cohen D., Ashkenazi S., Lerman Y., Egan W., Schneerson R., Robbins J. B. Synthesis, characterization, and clinical evaluation of conjugate vaccines composed of the O-specific polysaccharides of Shigella dysenteriae type 1, Shigella flexneri type 2a, and Shigella sonnei (Plesiomonas shigelloides) bound to bacterial toxoids. Infect. and Immunity. 1993, pp. 3678-3687), based on modification of exopolysaccharide by adipic dihydrazide (ADH) in the presence of 1-ethyl-3-(3-dimethylaminopropyl) carbodiimide (CDI) followed by reaction of the resulting amidated exopolysaccharide with a free hydrazide group with protein carrier—tetanus toxoid (TT).

Modification of exopolysaccharide with ADH in the presence of CDI were performed in water for 2-16 hours, keeping the pH between 4.8-5.2 by adding HCl concentrate with a pH-stat. Modified exopolysaccharide were separated on a column by Sephadex G-50 in water. Control of amidation levels was performed using C¹³-NMR spectroscopy. Conjugation of modified exopolysaccharide with tetanus toxoid carried out in 0.2 M sodium chloride solution in the presence of CDI for 4-18 hours, while maintaining pH 5.6 using the pH-stat. Conjugate was purified on column with Sepharose CL-6B from insignificant amounts of unconjugated biopolymers and impurities with low molecular weight, using 0.2M of sodium chloride solution as an eluent. Fractions, containing conjugate of the EPS with protein and eluted near the column void volume, were combined and phenol was added to a concentration of 0.05-0.15% for subsequent filling in sterile vials with addition of pharmaceutically suitable special additives, which include pH stabilizers or preservatives, or adjuvants, or isotonizing agents or combinations thereof.

The conjugate vaccine contained 40% protein mass, determined by Bradford method (Bradford M. M. A rapid and sensitive method for the quantitation of microgram quantities of protein utilizing the principle of protein-dye binding. Anal. Biochem. 1976, v. 72, pp. 248-254). One vaccination dose of conjugated vaccine contains: exopolysaccharide conjugate from 0.010 to 0.200 mg; phenol (preservative), not to exceed 0.75 mg, with the addition of sodium chloride, dibasic sodium phosphate and monobasic sodium phosphate; 0.5mL pyrogen-free sterile water for injection.

H. Conjugate Vaccine Immunogenicity

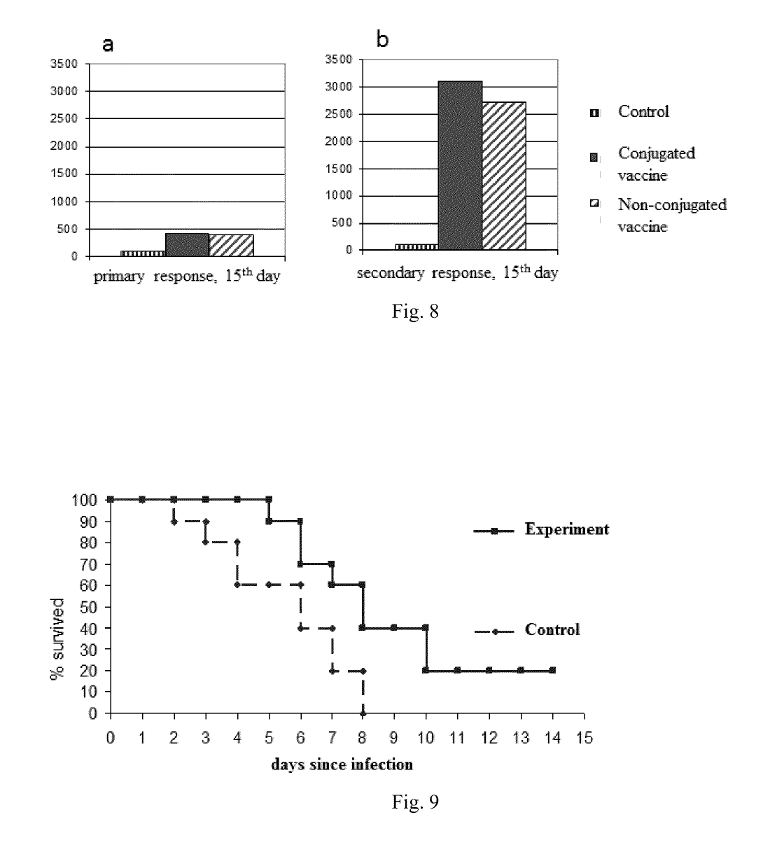

Two groups of mice (CBAXC57BU1/6) F1 were intraperitoneally immunized with a vaccine, includes unconjugated S.sonnei bacterial exopolysaccharide, lot 33 and a vaccine, includes conjugate S. sonnei bacterial exopolysaccharide, lot 33 with a TT carrier protein, at a dose of 25 mcg of polysaccharide per mouse. Unconjugated vaccine after a single dose immunization induces humoral immune response and 3.4-fold increase in IgG antibodies was detected at day 15 in the peripheral blood serum of animals. Conjugate vaccine also induces a humoral immune response after a single dose injection and 3.7-fold increase in IgG antibodies was detected at day 15 in the peripheral blood serum of animals at day 15 in peripheral blood serum of animals (FIG. 8).

To study secondary immune response the same groups of mice are vaccinated again with a dose of 25 mcg of polysaccharide per mouse a month after primary injection. On day 15 of the secondary response after second immunization with conjugate vaccine 27-fold rise of 1gG anti-O antibodies was registered, and after the second immunization with unconjugated vaccine—23.6-fold rise of IgG anti-O antibodies, respectively. Under this experiment the levels of O-specific antibodies significantly exceed the primary immune response antibody levels in immunized mice (FIG. 8B).

### Example 3

Pharmaceutical Composition Comprising S. sonnei, Phase I bacterial Exopolysaccharide

A. Use of the Exopolysaccharide for Production of Pharmaceutical Compositions (Pharmaceuticals)

Preparation a pharmaceutical composition includes obtaining the exopolysaccharide using S.sonnei, phase 1 bacteria in accordance with Example 1 (A) and subsequent filling into sterile vials or syringes of solution containing the active substance and a pharmaceutically suitable special additives, which can include preservatives, stabilizers, solvents, or a combination thereof.

Therapeutic dose of a pharmaceutical composition contains: exopolysaccharide, from 0.010 to 5,000 mg, with the addition of sodium chloride, dibasic sodium phosphate and monobasic sodium phosphate, 0.5 mL sterile pyrogen-free water for injection.

B. The Antiviral Effect of Pharmaceutical Compositions

Two groups of mice (CBAXC57B1/6)F1, 10 animals each, were infected with LD100 dose of virulent strain of influenza A subtype H1N1, after which the experimental group was treated with daily intraperitoneal administration of pharmaceutical composition to animals at a dose of 100 mcg of exopolysaccharide per animal; the control group of animals were similarly injected with saline. Animal survival rate was determined in the two weeks after infection. In the control group, the survival rate was 0%, in the experimental group—20% (FIG. 9). The mean survival time of the experimental group of mice was statistically significantly (p<0.001) higher than for mice of the control group. Thus, the experimental data show that the claimed pharmaceutical composition has a modulating effect on immune response.

1. Shigella sonnei, phase I polysaccharide, consisting of 1-100 repeating disaccharide units of O-[4-amino-2-(N-acetyl)amino-2,4-dideoxy-β-Dgalactopyranosyl]-(1->4)-O-[2-(N-acetyl)amino-2-deoxy-α-L-altrpyranuronic acid] linked within the polysaccharide chain by (1->3) bonds, produced using S. sonnei bacteria, but without using lipopolysaccharides as the source of production.

2. The polysaccharide according to claim 1, produced using S. sonnei bacteria by a method including: (a) production of the bacterial culture in liquid phase; (b) separating of the liquid phase from bacterial cells; (c) isolating of the polysaccharide from liquid phase.

3. The polysaccharide according to claim 2, produced using S. sonnei bacteria by a method, wherein during separation of the liquid phase from bacterial cells, a nativity of the bacterial cells is maintained.

4. The polysaccharide according to claim 2, produced using S. sonnei bacteria by a method, wherein isolating of the polysaccharide from the liquid phase includes: (i) removal of proteins and nucleic acids from the liquid phase; (ii) ultrafiltration, and (iii) dialysis of the obtained solution.

5. The polysaccharide according to claim 1, in the form of a exopolysaccharide, secreted by S. sonnei, phase I bacteria into external medium.

6. The polysaccharide according to claim 5, in the form of a bacterial capsular polysaccharide.

7. The polysaccharide according to claim 1, containing a non-toxic lipid component.

8. The polysaccharide according to claim 7, wherein non-toxic lipid component includes non hydroxylated fatty acids with 16-18 carbon atoms per molecule.

9. (canceled)

10. The polysaccharide according to claim 1, having molecular weight, measured by gel-filtration method, from 0.4 to 400 kDa.

11. The polysaccharide according to claim 1, containing no more than 1 (w/w) percent of protein and 2 (w/w) percent of nucleic acids.

12.-13. (canceled)

14. A method for the production of polysaccharide according to claim 1, which includes: (a) producing cultures of S. sonnei bacteria in liquid phase; (b) separating of the liquid phase from bacterial cells; (c) isolating of the polysaccharide from liquid phase.

15. The method according to claim 14, wherein during separation of the liquid phase from bacterial cells, the nativity of the bacterial cells is maintained.

16. The method according to claim 14, wherein the isolating of the polysaccharide from
the liquid phase includes: (i) removing of proteins and nucleic acids from the liquid phase; (ii) ultrafiltration, and (iii) dialysis of the obtained solution.

17.-50. (canceled)

51. A use of the polysaccharide according to claim 1 for production of pharmaceutical composition.

52. The use according to claim 51, wherein the polysaccharide is produced using S. sonnei bacteria by a method, including: (a) production of the bacterial culture in liquid phase; (b) separating of the liquid phase from bacterial cells; (c) isolating of the polysaccharide from liquid phase.

53. The use according to claim 52, wherein the polysaccharide is produced using S. sonnei bacteria by a method, which includes separation of the liquid phase from bacterial cells while maintaining a nativity of the bacterial cells.

54. The use according to claim 52, wherein the polysaccharide is produced using S. sonnei bacteria by a method, which includes the following stages for its isolation from a liquid phase: (i) removing of proteins and nucleic acids from the liquid phase; (ii) ultrafiltration, and (iii) dialysis of the obtained solution.

55.-56. (canceled)

57. The use according to claim 51, wherein the polysaccharide has a nontoxic lipid component.

58. The use according to claim 57, wherein the polysaccharide has, as a non-toxic lipid component, non hydroxylated fatty acids having 16-18 carbon atoms per molecule.

59. The use according to claim 58, wherein the amount of non hydroxylated fatty acids in the polysaccharide is no less than 0.01 (w/w) percent.

60. The use according to claim 51, wherein the polysaccharide has molecular weight, measured by gel-filtration method, from 0.4 to 400 kDa.

61. The use according to claim 51, wherein the polysaccharide contains no more than 1 (w/w) percent of protein and 2 (w/w) percent of nucleic acids.

62.-64. (canceled)

65. The use according to claim 51, wherein the vaccine or pharmaceutical composition are intended for parenteral, oral, rectal, intra-vaginal, transdermal, sublingual, and aerosol administration to mammals, including humans.
